(19) United States
(12) Patent Application Publication
(10) Pub. No.: US 2025/0204874 A1
(43) Pub. Date: Jun. 26, 2025

(54) IMAGING SYSTEM

(71) Applicant: Elekta Limited, Crawley (GB)

(72) Inventors: Markus Eriksson, Gustavsberg (SE); Stefan LANGEMARK, Crawley (GB)

(21) Appl. No.: 18/990,372

(22) Filed: Dec. 20, 2024

(30) Foreign Application Priority Data
Dec. 22, 2023 (GB) 2319966.4

(51) Int. Cl. A61B 6/40; A61B 6/00; A61B 6/03; G01T 1/20

(52) U.S. Cl. CPC A61B 6/4085; A61B 6/032; A61B 6/4435; A61B 6/5205; G01T 1/20182

(57) ABSTRACT

An imaging system can comprise a source of radiation rotatable around a subject and configured to deliver imaging radiation to the subject from a plurality of angles, and detector comprising a plurality of rows of detector elements. The system is configured to receive the radiation, read out image data representing received radiation successively from each row of the detector, assign a rotation angle to each row of the image data, and output each row of image data and the respective assigned rotation angle to an image reconstruction algorithm for reconstruction of a three-dimensional image of the subject.

### CLAIM FOR PRIORITY

This application claims the benefit of priority of British Application No. 2319966.4, filed Dec. 22, 2023, which is hereby incorporated by reference in its entirety.

### TECHNICAL FIELD

This disclosure relates to cone-beam computed tomography (CBCT), specifically to CBCT using a continuous beam and methods therefor.

### BACKGROUND

Radiotherapy can be described as the use of ionising radiation, such as X-rays, to treat a human or animal body. Radiotherapy can be used to treat tumours within the body of a patient or subject. In such treatments, ionising radiation is used to irradiate, and thus destroy or damage, cells which form part of the tumour. Before radiation treatment, a radiotherapy treatment plan can be created to determine how and where the radiation should be applied. Such a treatment plan can be created with the assistance of medical imaging technology. For example, a computed tomography (CT) scan may be taken of the patient in order to produce a three-dimensional (3D) image of the area to be treated. The 3D image allows the treatment planner to observe and analyse the target region and identify surrounding tissues. In addition, a series of 3D images can be recorded over a period of time, such as a breathing cycle, in order to provide a four-dimensional (4DCT) video that can inform the treatment plan. These images can be acquired using a dedicated imaging device such as a CT scanner. Such devices allow the patient to be imaged on the day of treatment, for example to ensure their anatomy is properly aligned with the anatomy shown in the images forming the basis of the treatment plan. Images may also be taken during treatment to improve the precision and accuracy of the treatment.

### SUMMARY

Imaging systems may include CT imaging systems and cone beam computed tomography (CBCT) imaging systems. Radiotherapy devices may also comprise such imaging systems. CBCT uses a cone-shaped beam and detector panel to cover a large volume of a subject with each rotation around the subject, thereby acquiring image data of the subject from various angles. An area detector is used to detect the cone beam. The image data is then used to reconstruct a 3D image. CBCT systems are therefore able to provide 3D images quickly, with a reduced number of rotations and a lower dose of radiation in comparison to traditional CT systems. For example, it is possible to achieve a 3D imaging volume via a single 180 degree rotation around a subject using CBCT modality.

While computed tomography (CT) operates with a continuous imaging beam, traditionally CBCT uses pulsed kilovolt (kV) radiation. In CBCT, during rotation of the x-ray source about the subject, the subject is radiated with many short radiation pulses. A flat panel x-ray detector receives the radiation behind the subject. Directly after the pulse of radiation, the detector pixels are read out to form a projection image. The source is pulsed and the data is read out sequentially. When the source has moved a delta angle, the next x-ray pulse is given. This is completed until the whole source rotation is completed.

There is a limitation as to how short a radiation pulse can be generated, meaning there will be a speed limit to the rotation of the source at which the imaging pulses are ‘back-to-back’. This source rotation speed limit associated with pulsed radiation necessitates long image acquisition times. A long acquisition time may be uncomfortable for the patient, and gives a high probability of patient motion during image acquisition. Patient motion may involve, for example, a patient not lying still or gas movements in the bowel. Patient motion causes image artefacts, or image defects, which adversely affect image quality and can cause problems when using the image. For example, the image may be used in registration, organ segmentation or treatment dose calculation and therefore artefacts or defects in an image could cause, for example, incorrect treatment dose calculation. As a principle, to reduce motion artifacts, the imaging should be performed in the shortest time as possible. Accordingly, there is a need to reduce image acquisition times.

The present disclosure relates to an imaging system comprising a source of radiation rotatable around a subject and configured to deliver imaging radiation to the subject from a plurality of angles. A detector comprising a plurality of rows of detector elements can be configured to: receive the radiation; read out image data representing received radiation successively from each row of the detector; assign a rotation angle to each row of the image data; and output each row of image data and the respective assigned rotation angle to an image reconstruction algorithm for reconstruction of a three-dimensional image of the subject.

In an example, the detector may be rotatable in synchrony with the source. The source may be configured to continuously deliver the radiation while rotating through the plurality of angles. The source may be configured to continuously rotate through the plurality of angles. Each row may be assigned its own respective angle. The rotation angle assigned to each row of data may be different. The rotation angle assigned to each row of data may be different than the angle assigned to the previously read-out row of data.

The image data may form a first set of image data and the detector is configured to, once the first set of image data has been read out, receive subsequently delivered radiation and read out image data representing the subsequently received radiation successively from each row of the detector to form a second set of image data.

The detector may be configured to read out a plurality of sets of image data, wherein each set of image data comprises a plurality of rows. The source may be configured to rotate though 360 degrees around the patient.

The radiation may comprise a cone-shaped beam of radiation. The radiation may comprise x-ray radiation. The rotation angle may correspond to a particular angular position, or angular range, of the source around the subject. The detector may be positioned diametrically opposite the source. The source and detector may be coupled to a rotatable gantry. The system may further comprise an image reconstruction system configured to reconstruct the three-dimensional image based at least in part on each of the rows of image data and the respective assigned rotation angles.

There is also provided a method for imaging a subject using the imaging system as disclosed above. The method may comprise receiving, at a detector comprising a plurality of rows of detector elements, radiation delivered from a source to a subject; reading out, from the detector, image data representing the received radiation successively from each of the plurality of rows; assigning a rotation angle to each row of the image data; outputting each row of image data and the respective assigned rotation angles to an image reconstruction algorithm for reconstruction of a three-dimensional image of the subject. The method may further comprise rotating the source around a subject and delivering imaging radiation to the subject from a plurality of angles.

The method may further comprise rotating the detector, in synchrony with the source, around a subject. Optionally, the source is configured to continuously deliver the radiation while rotating through a plurality of angles. The image data may form a first set of image data and, wherein once the first set of image data has been read out, the method may further comprise receiving subsequently delivered radiation and reading out image data representing the subsequently received radiation successively from each of the plurality of rows to form a second set of image data.

Obtaining image data may comprise obtaining a plurality of sets of image data, wherein each set of image data comprises a plurality of rows. The radiation may comprise a cone-shaped beam of radiation and/or the radiation comprises x-ray radiation.

The rotation angle may correspond to a particular angular position, or angular range, of the source around the subject. The detector may be positioned diametrically opposite the source. The source and detector may be coupled to a rotatable gantry. The method may further comprise rotating the gantry about the subject. Reconstruction of the three-dimensional image may be based at least in part on each of the plurality of rows of image data and the respective assigned rotation angles.

### BRIEF DESCRIPTION OF THE FIGURES

Specific embodiments are now described, by way of example only, with reference to the drawings, in which:

FIGS. 1A and 1B depict examples of a CBCT imaging system according to the present disclosure;

FIG. 2 depicts an example of a detector panel for a CBCT imaging system according to the present disclosure;

FIG. 3 depicts an example of a method according to the present disclosure;

FIG. 4 depicts an example of an imaging system according to the present disclosure;

FIG. 5 depicts an example of a computer program product according to the present disclosure.

### OVERVIEW

It is desirable to image a patient in the shortest possible time for the reasons given in the background section. CBCT uses pulsed imaging radiation. There is a limitation as to how short a radiation pulse can be generated, meaning there will be a speed limit to the rotation of the source at which the imaging pulses are ‘back-to-back’. This source rotation speed limit associated with pulsed radiation necessitates long image acquisition times in CBCT.

Using a continuous beam can allow for fast gantry rotation and can reduce the acquisition time of an image. Image acquisition times can be reduced by removing the rotation speed limitation associated with pulsed radiation, in particular by using a continuous imaging beam. Hence, there is a need to provide CBCT imaging methods and systems using a continuous rather than pulsed beam. The present disclosure uses a continuous beam in CBCT imaging to reduce image acquisition times.

### Delivering a Continuous Beam and Continuously Reading Out Image Data Form Pixels Row by Row

A CBCT flat panel running in continuous read out mode reads the detector row by row during rotation and radiation. In pulsed imaging the panel is read out after the pulse of radiation. For each read-out of the panel, the detected radiation read out from the pixels are all received from the same pulse, delivered at the same angle (or same small delta angle).

In continuous beam imaging, where the panel runs in continuous read-out mode, the first row of the detector is radiated during one angular range of radiation and the next row a delta increment later and so on until all rows are read and continuously starting over with the first row again. A projection image of rows will thereby have a skewness from which angular range the different rows are formed. This will in turn give a skewness in the reconstructed 3D image formed by these images. A skewness in the reconstructed 3D image will reduce the image accuracy and quality and adversely affecting analysis of the images for e.g. treatment planning.

The system and method in present disclosure reduces imaging time while providing an accurately constructed 3D image without skew. Each row of imaging data is assigned a different imaging angle corresponding to the angle at which the detected radiation being read out was delivered.

An imaging system and method for imaging a subject are provided. Radiation is delivered continuously from a source rotatable about the subject, and imaging data is obtained at a detector rotatable about the subject. A continuous cone beam of radiation is used to carry out fast acquisition of projection data. The imaging data comprises a plurality of rows. A rotation angle is assigned to each row of the image data, and the detector reads out each row of the image data successively. The assigned rotation angle may be different for each row of image data, to account for the continuous rotation of the source. Each row of the image data and the respective assigned rotation angles are delivered to an image reconstruction algorithm for reconstruction of a three-dimensional image of the subject. This means that the rotation of the source around the subject during delivery of the continuous beam can be accounted for, and skewness in the reconstructed 3D image can be mitigated.

By assigning a rotation angle to each row of imaging data, the image data can accurately be reconstructed onto an image since the assigned angle accurately reflects that rotation angle at which the radiation was received. It enables continuous delivery of the radiation beam. When the image is reconstructed there is no skewness to the image since each row is processed with its own rotation angle. Therefore an accurate reconstruction of the image is provided with a shorter exposure time.

### DETAILED DESCRIPTION

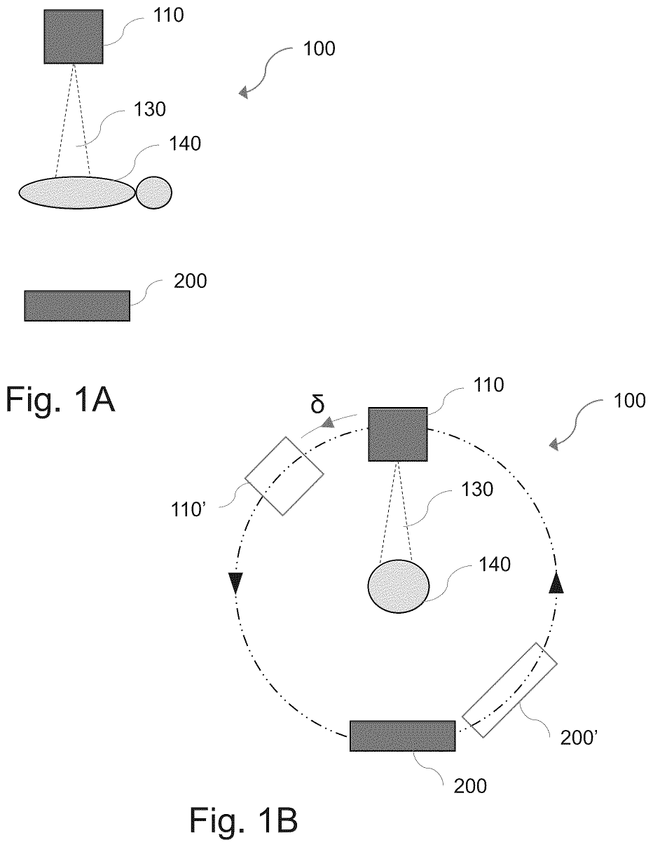

FIG. 1A depicts a CBCT imaging system 100 according to the present disclosure from a first perspective. FIG. 1A shows a side-on view of an imaging system. The imaging system is configured to image a subject 140. The imaging system 100 comprises a source of imaging radiation 110, such as a source of x-ray radiation, and an imaging detector 200. The source 110 is configured to emit a continuous beam of radiation 130, such as a continuous beam of x-ray radiation, towards the subject 140. Detector 200 may also be referred to herein as a detector panel or panel or imaging panel or flat panel. The source 110 and detector are configured to rotate around the patient, the axis of rotation shown as A in the plane of the diagram. The source may be configured to continuously rotate, and/or continuously deliver radiation while rotating.

FIG. 1B depicts the CBCT imaging system 100 according to the present disclosure from a second perspective. FIG. 1B shows an end-on view of the imaging system shown in FIG. 1A. FIG. 1B is taken at an angle of 90 degrees relative to FIG. 1A.

The source 110 and detector 200 are rotatable around or about the subject 140, as indicated by the dashed line in FIG. 1B. FIG. 1B depicts the source 110 and detector 200 in an initial starting position as shown in FIG. 1A, and also shows the source and detector rotated about the subject 140 by an angular increment δ, indicated by rotated source 110′ and rotated detector 200′. The axis of rotation in the example embodiment of FIG. 1B is shown by A which perpendicular to the plane of the diagram (i.e. comes out of the plane of the diagram). Upon rotation about the subject 140, the source 110 and detector 200 remain arranged with respect to one another such that the beam of radiation 130 emitted from the source can always be detected at the detector panel 200. In particular, the source 110 and detector 200 may be arranged diametrically opposite one another, and remain diametrically opposite one another during rotation about the subject 140.

By maintaining the source 110 and the detector 200 in a diametrically opposite relationship, the detector is always positioned in the beam path 130. Radiation which is emitted from the source 110 passes through the patient 140 and is detected by the detector 200. Thus the source 110 and the detector 200 are rotated at the same rate around the patient and maintain a diametrically opposing relationship.

In some embodiments, one full rotation of the source 110 and detector 200 about a subject 140 may comprise 360 degrees. The source 110 and detector 200 may be arranged such that they are, and remain during rotation, separated by 180 degrees. In other words, the detector may be rotatable in synchrony with the source.

In some embodiments, the source 110 and/or detector 200 may be coupled to a rotatable gantry (not shown), such that rotating the gantry about the subject 140 comprises rotating the source 110 and detector 200 about the subject 140. The source 110 and/or detector 200 may be fixedly attached to the gantry, optionally with the source positioned diametrically opposite the detector 200. The source 110 may remain diametrically opposite the detector 200 during rotation of the gantry about the subject 140.

In some embodiments, a radiotherapy device (not shown) may comprise the CBCT imaging system 100. The radiotherapy device may also comprise a source of therapeutic radiation for delivering a treatment beam, as well as other devices and constituent components well known to the skilled person. For example, the radiotherapy device may comprise a source of radiofrequency (RF) waves, RF transmission apparatus, an accelerating waveguide, a source of electrons, a treatment head including a collimator such as a multi-leaf collimator used to shape a treatment beam, other beam-shaping devices, a housing and/or a patient support surface for supporting a patient subject to radiotherapy and/or imaging.

The imaging system 100 is configured for CBCT imaging. As will be familiar to those skilled in the art, the source of imaging radiation 110 is configured to emit a cone-shaped beam 130, having a cross-section having a similar beam width in each dimension.

During traditional CBCT imaging, the source of radiation rotates about a subject, taking ‘snapshot’, or projection, images at various angles which can be used to produce a 3D image of the subject. For each snapshot image, the source delivers a pulse of imaging radiation which passes through the subject and reaches the detector. Image data is obtained at pixels across the detector panel, and the image data can be read out. The source and detector are rotated continuously while the source of radiation is pulsed. Each ‘snapshot’ image represents radiation which is delivered and received in a small delta angle (during the short pulse). After the pulse is delivered, data is read from the detector. The data corresponds to the radiation delivered and detected at the same angle or at the same small delta angle. The source and detector continue to rotate and another pulse is delivered to obtain the next snapshot image, and so on. Each snapshot image, therefore, corresponds to a single angular position (or small delta angular position) of the source of imaging radiation.

The imaging radiation has an energy suitable for imaging a patient. The systems and methods disclosed herein may include imaging radiation. Some embodiments of the system and method disclosed herein relate to non-therapeutic radiation.

However, in the present disclosure the source 110 is configured to emit a continuous beam of radiation, as opposed to a pulse beam as in traditional CBCT imaging systems. The source 110 and detector 200 can be continuously rotated about the subject 140 during continuous delivery of the beam, according to the position and speed requirements necessary to produce the desired image. As the source 110 and detector 200 are rotated about the subject 140, image data is obtained or acquired at the detector 200 and read out.

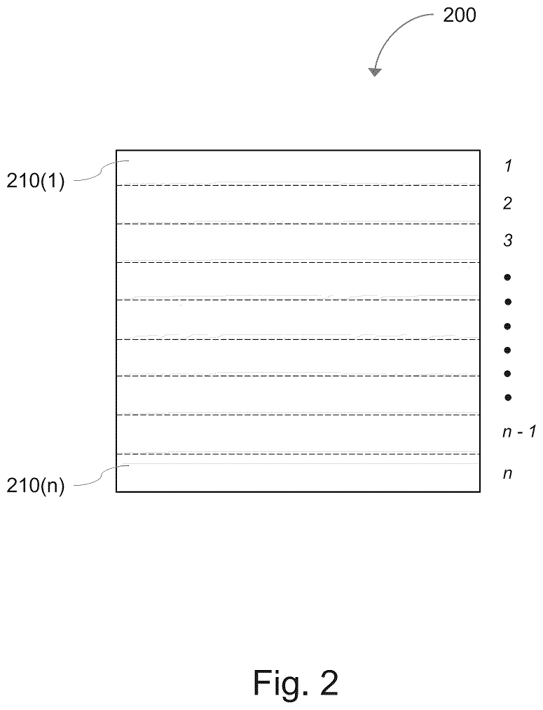

FIG. 2 depicts a detector panel 200 for imaging according to the present disclosure. The detector panel 200 may be referred to herein as a detector or panel or imaging panel or flat panel. The detector panel is an area panel, in that is have two dimensions of pixels (as opposed to a single dimension, i.e. a line or single row of pixels). The detector panel 200 is rotatable about the subject. The panel 200 comprises a plurality of rows 210 of pixels or detector elements (not shown). In the embodiment shown FIG. 2, the plurality of rows comprises n rows 210(1)-210(n). Detector 200 is configured to receive and/or detect radiation, in particular the radiation delivered from source 110.

Radiation is emitted from the source and passes through the subject 140. Once radiation emitted from the source 110 has passed through the subject 140, the radiation continues towards detector 200, where it is blocked/absorbed. As the radiation passes through the patient it is absorbed by the patient's body. Radiation is absorbed to a different degree by the patients' organs and bones. The density and volume of the tissue and bone through which the radiation travels affects how much radiation is absorbed by the patient. The intensity of the radiation leaving the patent therefore includes information about the internal organs in the patient. By measuring the intensity of radiation which has passed through the patient, an image of the internal organs of the patient can be determined.

The detector 200 may comprise or include an imaging panel. The detector 200 may be configured to produce signals indicative of the intensity of radiation incident on the detector 200. In use, these signals are indicative of the intensity of radiation which has passed through the subject 140. These signals may be processed to form an image of the subject 140. This process may be described as the imaging apparatus and/or the detector 110 capturing an image or obtaining image data. The image data represents radiation received at the detector. By taking images, i.e. obtaining image data, at multiple angles around the subject 140 it is possible to produce a 3D image of the patient. The detector 200 may form part of an electronic portal imaging device (EPID). EPIDs are generally known to the skilled person and will not be discussed in detail herein.

The detector 200 may comprise a flat panel imager, which may comprise a scintillator. Radiation incident on the scintillator will produce light. The flat panel imager may comprise an array of photodiodes and transistors, each corresponding to a particular pixel of the detector/flat panel imager. The light from the scintillator impinging on the photodiodes creates respective electronic signals which are gated by the respective transistors. These electronic signals are extracted from the flat panel array via read-out electronics to form a digital data stream that is used to construct an image. Generally, the pixel elements of such detectors work by outputting a respective signal in which the total charge passed reflects the total incident radiation since the last time the pixel was read. As radiation is incident on the pixel, it causes ionisation and the resulting charge is retained. When the pixel is enabled, i.e. when it is triggered to release its signal, that charge is output to be counted. The flat panel imager may comprise an interpreter configured to receive the signal outputs. The interpreter may comprise an integrator configured to integrate the signal outputs to measure the charges collected at the respective pixels and thus provide an indication of the radiation received by the pixels of the flat panel imager. This can be used to identify the shape and location of objects (e.g. the subject) between the source and detector through the relative lack of radiation received at the pixels for which the radiation from the source was blocked by the object.

The pixels of the detector may be arranged in a rectilinear manner with the pixels in straight rows 210 and columns. The intersection of a particular row with a particular column therefore defines a specific pixel. Each column may have a common output line which allows the charge that has accumulated on each pixel to escape to the integrator where it is multiplexed with the outputs of other columns. This may enable the entire line of pixels to be read out at the same time.

The image data obtained at the detector 200 comprises a plurality of rows, which correspond to rows of the detector 200. In other words, obtaining image data at the detector 200 may comprise obtaining a plurality of rows of image data. The detector 200 is configured to read out obtained image data, and may comprise scanning control electronics which enable each row to be read successively, with the whole row read at substantially the same time. The integrator is then reset, and the next row is enabled. Thus, the detector is configured to read out each row of image data successively.

The detector may be described as reading out the image data in a continuous manner, such that there is minimal or no temporal break between the successive reading out of each row of image data. In other words, the detector may read out image data continuously or substantially continuously. This may be referred to as a continuous readout mode of the detector 200.

In continuous readout mode, the detector reads out the first row of the image data, followed by the second row of the image data, followed by the third row of the image data etc for all rows of the image data. A readout of image data for all rows successively may be referred to as a full panel readout. After a first full panel readout, the detector may restart readout again for a second set of obtained image data. In particular, after a first full panel readout, the detector may then start reading out a first row of the second set of image data, followed by a second row of the second set of image data, etc. This may be referred to as a second full panel readout. The detector may perform two or more successive full panel readouts. Again, as used herein successive means one after the other such that the detector may commence one full panel readout after completion of a previous full panel readout. Successive full panel readouts may be performed in a continuous manner. Again, as used herein, continuous means with minimal or no temporal break between full panel readouts.

In traditional CBCT the radiation is pulsed. All the rows of the detector panel are read out after the pulse, reading out imaging data which was received at the panel during the pulse (i.e. a ‘snapshot’ image of the gantry at the specific small delta angle of rotation the source was at when the pulse was emitted). Incidentally, in the pulsed configuration all of the rows of detector elements are reading out imaging data which was received at the same small delta gantry angle, and therefore reading out rows sequentially does not affect or skew the data.

Between each pulse, a full readout of the panel is performed. Therefore each snapshot image, i.e. the image data obtained from each pulse of radiation and full read out of the detector, corresponds to a single angular position or small delta angle of the source/detector around the subject. A delta angle is a segment of the arc moved through by the source. In normal CBCT, all rows of image data obtained for a given pulse of radiation correspond to the same angle of rotation (or small delta angle) of the source/detector around the patient. Therefore, each projection image corresponds to a single angle of rotation of the source/detector around the patient. All rows measure the same angle or delta angle.

However, in the present disclosure this is not the case. In the system disclosed herein, the source 110 is configured to emit a continuous beam of radiation, as opposed to a pulse beam. Additionally, the source 110 and detector 200 rotate continuously around the subject delivering the continuous beam 130 towards the subject 140. Therefore, radiation is continuously received and detected at the detector 200 at all angles of rotation around the patient. This means that radiation received at the detector 200 will have passed through the subject 140 at source 110 and detector 200 positions varying continuously through all angles of the rotation.

When the radiation is received, image data is obtained at the detector 200. Obtaining the image data may comprise receiving or detecting the radiation. The radiation may be received or detected at each row of the detector, optionally by the one or more pixels.

During image acquisition, the source and detector rotate about the subject until a full (360 degree) or half or other partial rotation is completed. The received or detected radiation has passed from the source 110 through the subject 140, and since the source 110 and detector 200 rotate about the subject 140, radiation will be received which corresponds to all angles in the range through which the source rotates. The radiation received at the detector 200 has passed through the subject 140 at the angle which the source/detector are relative to the patient when the radiation is emitted. The radiation received gives information about the internal organs in a patient from the specific angle of the source.

As the source and detector rotate, the imagining data is read out continuously as explained above. That is, each row of data is read out in order. The radiation, rotation and read-out are all continuous. The readout of the rows of imaging data means that there is a time difference between each row of data being read out. In this time the source and detector have rotated around the patient. This means that at each time a row of data is read out from the imaging detector, the position of the source/detector has changed. Data which is read out from a given row of the detector relates to radiation which has passed through the patient at a specific angle, and this angle is different to the angle of data read out from a different row of the detector.

This means that in some embodiments the first row 210(1) of the detector is radiated by the continuous beam during one angular range of source position, and the next row 210(2) is radiated during a second angular range of source position. The first and second angular ranges may differ by an angular increment δ, i.e. the source and detector may move through an angle δ between delivering the radiation which is eventually received at the first and second rows of the detector respectively. The third row 210(3) is radiated during a third angular range of source position which may differ from the second angular range, and so on for all n rows. In other words, each of the rows of image data corresponds to particular angular source 110 position at which the radiation, received at the detector in the obtaining of that row of image data, was delivered to the detector 200.

Once image data has been obtained, the projection images can be used in image reconstruction to form a 3D image of the patient. Using traditional CBCT imaging techniques, each of the snapshot, or projection, images taken with each pulse of radiation gives information about the internal structure and organs of the patient from a particular angle. These projection images are then used in image reconstruction to form the 3D image which gives a 3D view of said internal structure and organs of the patient. Since each snapshot image is taken at a single angle with the source and detector stationary during the pulse of radiation, the angle at which the radiation passed through the subject does not change during acquisition of each snapshot image. This means that each snapshot image corresponds to a well-defined particular perspective of the patient, and therefore skewness in the 3D reconstructed image is avoided.

On the other hand, when using CBCT imaging systems with a continuous beam, as explained above the source and detector rotate continuously around the subject during continuous radiation of the beam. Radiation is received and image data is acquired at the detector corresponding to all angles around the subject. Therefore, the image data for each projection image does not necessarily correspond to a single particular perspective of the patient, and thus a projection image formed from the rows of image data will have a skewness as a result of the difference 8 in angular range of the source position corresponding to each of the n rows. This in turn will lead to skewness and potentially other inaccuracies in the reconstructed 3D image.

As described above, the use of a continuous beam allows for shorter image acquisition times compared to traditional CBCT imaging using a pulsed beam. This in turn reduces the probability of patient motion during imaging, thereby reducing image artefacts and improving image quality. Shorter image acquisition times are also more comfortable and pleasant for the patient.

Furthermore, the use of a continuous beam improves the lifetime of the source of imaging radiation, and is more efficient than the use of a pulsed beam both in terms of power consumption as well as computational resources used to control the beam.

The continuous readout mode of the detector 200 described above enables continuous data readout by the detector during continuous rotation and radiation of the source and imaging beam. In other words, the detector 200 is configured for use in CBCT imaging with a continuous imaging beam. A computer-implemented method suitable for use in conjunction with a detector according to any embodiments described herein will now be set out.

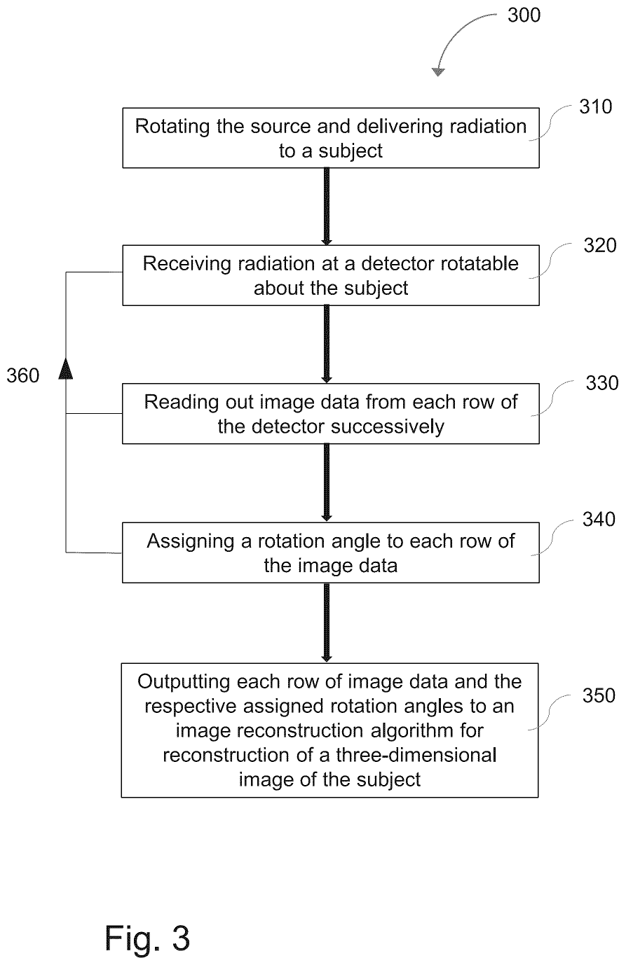

FIG. 3 depicts a method 300 according to the present disclosure. In particular, FIG. 3 depicts a method 300 for imaging a subject. The method 300 can be used with imaging system 100 comprising detector panel 200. In some embodiments the method 300 may be used with a radiotherapy device comprising an imaging system such as system 100 comprising a detector panel 200. The method of FIG. 3 can be used to address the issue of skewness with a continuous beam and continuous rotation discussed above.

In an embodiment, a method 300 comprises rotating a source 110 of radiation about a subject, and delivering 310 radiation from the source 110 to the subject. The radiation is delivered to a detector 200 configured to detect or receive the radiation from the source. The detector is also rotatable about the subject. The method 300 comprises receiving 320 the radiation at the detector 200. In other words, the radiation is delivered during rotation of the source 110 about the subject, and the detector 200 receives the radiation during rotation of the detector 200 about the subject. Image data is obtained by the detector 200 upon reception of the radiation, as explained in detail with reference to FIG. 2.

The detector 200 comprises a plurality of rows of detector elements, and is configured to read out the image data of the received radiation. In particular, the detector is configured to read out image data successively from each of the plurality of the rows. In other words, the detector 200 is configured to read out the image data row by row. The image data may be described as comprising a plurality of rows, each of which may correspond to a particular row of detector elements.

The method 300 comprises reading out 330, from the detector, image data of the received radiation successively from each of the plurality of rows of detector elements. As described in detail with respect to FIG. 2, the detector 200 operates in a continuous readout mode. In other words, the detector reads out image data from each row successively, in a continuous manner. This means that data is continuously read out by the detector, row-by-row, which ensures fast and efficient readout of the data.

In some embodiments the source 110 and/or detector 200 are coupled to a rotatable gantry, such that the radiation is delivered, and/or the image data is obtained, during rotation of the gantry. The source and detector are positioned diametrically opposite one another, such that the detector remains opposite the source during rotation. All embodiments described herein may be suitable for use in systems wherein the source is coupled to a rotatable gantry, though other rotation mechanisms known to the skilled person may also be used such as arms.

In embodiments described herein, delivering radiation from a source may comprise delivering a continuous beam of radiation from the source. In embodiments for use with CBCT, the beam is cone-shaped. The radiation may comprise x-ray frequencies. The method may be applied to imaging using any type of radiation with a moving array that simultaneous reading out rows/pixels in one part of the detector while other parts are measuring by integration.

The method 300 comprises assigning 340 a rotation angle to each row of the image data. Each row is assigned its own respective angle. The detector is configured to assign the rotation angle to each row. The rotation angle corresponds to a particular angular position or angular range of the source around the subject at the time which the detected radiation was delivered. The rotation angle may be described as corresponding to, for a given row of the image data obtained by the detector, the angular position of the source at which the radiation was delivered. In other words, the rotation angle may relate to the angular position of the source when the source emitted the radiation detected to form the particular row of the image data obtained by the detector. For embodiments in which the source is coupled to a rotatable gantry, the rotation angle may also be referred to as a gantry angle.

The rotation angle may be determined with respect to a ‘zero’ or reference position. In an exemplar embodiment, one full rotation of the source about the subject may comprise 360 degrees and so each row of the image data may be assigned an angle between 0 and 360 degrees. In other embodiments, other angular reference systems may be used.

Image data is obtained for various positions of radiation source, i.e. image data is obtained which corresponds to various rotation angles. Each row of the image data is assigned a rotation angle, such that a full panel readout comprises image data corresponding to at least one angular source position. In exemplar embodiments, a full panel readout may correspond to a full rotation of the source about the subject, i.e. a range of rotation angles between 0 and 360 degrees. In other exemplar embodiments, a full panel readout may correspond to more than one full rotation of the source about the subject. In alternative exemplar embodiments, a full panel readout may correspond to a partial rotation of the source about the subject, i.e. a subset of the full range of rotation angles. For example, a full panel readout may correspond to a range of rotation angles between 0 and 180 degrees, or any other subset of rotation angles between 0 and 360 degrees. In tomosynthesis a range of 180 degrees may be used. In helical acquisition (CT) a range of greater than 360 degrees (i.e. greater than one complete rotation of the gantry) may be used. Reading out each row of image data successively, each row being assigned a rotation angle, enables the detector to provide continuous readout of image data while taking into account the rotation of the source about the subject during delivery of the continuous beam.

In embodiments where the source is coupled to a gantry, obtaining image data for all desired gantry angles comprises rotating the gantry about the subject, through one or more full rotation. Alternatively, obtaining image data for all desired gantry angles may comprise rotating the gantry about the subject through a partial rotation, i.e. rotation through a subset of the full range of gantry angles.

Since the source is being rotated about the patient, the assigned rotation angle may be different for each row of image data in a full panel readout. It can be envisaged that if a source were to be stationary doe the duration of image data being read out over multiple rows, then those image data rows would be assigned the same rotation angle. However for continuous rotation of the source, the source is in a different position when each row of image data is read out, and therefore each row of image data is assigned a different angle.

The method 300 comprises outputting 350 each row of the image data and the respective assigned rotation angles to an image reconstruction algorithm for reconstruction of a three-dimensional (3D) image of the subject. In other words, each row of the image data has an assigned rotation angle, and each row of data and its assigned rotation angle are delivered to an image reconstruction algorithm. The detector is configured to output the image data and the respective assigned rotation angles to the algorithm.

The algorithm uses each row of the image data and the respective assigned rotation angles to reconstruct a 3D image of the subject or part of the subject. In other words, the reconstruction of the three-dimensional image of the subject is based at least in part on the image data and the rotation angles assigned to each row of the image data. In some embodiments, the image reconstruction algorithm may additionally or alternatively reconstruct one or more two-dimensional images of the subject or part of the subject.

In the acquisition, each pixel measures the attenuation in the object along a line between the source and the pixel. The purpose of the image reconstruction algorithm is to create an image based on these measurements. The pixel values are filtered, and image processed before they are back projected. The back projection takes the processed pixel information and distributes it along a line that geometrically corresponds to the measured line.

Accordingly it is important that the position/gantry angle of source when the radiation emitted/detected is a the pixel is correct. If the assigned position deviates this leads to a blurring effect, which can cause geometric inconsistencies or artefacts in the reconstructed image.

Providing an image reconstruction algorithm with not only image data but also angles representing the corresponding source position for each row of the image data enables the algorithm to take into account the rotation of the source about the subject during delivery of the continuous imaging beam. This means that skewness in the projected image and hence in the reconstructed 3D image can be mitigated or eliminated entirely. Images with reduced skewness are more geometrically accurate and of higher quality, which in turn reduces the risk of incorrect analysis or interpretation of the image during, for example, adaptation of treatment plans or other clinical workflows.

The present methods therefore provides correct imaging geometry of a patient for all IGRT treatments.

The steps of method 300 described herein of receiving radiation 320, reading out image data from each row 330 and/or assigning a rotation angle 340 may be repeated 360 until sufficient image data has been obtained, assigned an angle and read out as needed. Exemplar embodiments of the method 300 in which steps 320, 330 and/or 340 may be repeated 360 will now be described.

The image data obtained at the detector according to method 300 may be, or form, a first set of image data. The method 300 may further comprise receiving, at the detector, subsequently delivered radiation. The subsequently delivered radiation may be received as the source is continuously rotating about the subject, and so may correspond to different rotation angles to those assigned to each row of the first set of image data. The method may further comprise reading out image data representing the subsequently received radiation successively from each row of the detector, to form a second set of image data.

For example, radiation may be received 320 at the detector, and a first set of image data of the received radiation may be read out 330 from the detector successively from each of the plurality of rows. Radiation may then be received 320 again at the detector, and a second set of image data of the received radiation may be read out 330 after all of the rows of the first set of image data have been read out. In other words, the reading out of the first set of image data may be referred to as a first full panel readout, and/or the reading out of the second set of image data may be referred to as a second full panel readout, optionally wherein the second full panel readout commences after completion of the first full panel readout.

The method 300 may comprise assigning 340 a rotation angle to each row of the first set of image data, and assigning 340 a rotation angle to each row of the second set of image data. The method 300 may further comprise outputting 350 each row of the second set of image data and their respective assigned gantry angles, as well as the first set of image data and respective assigned gantry angles, to the image reconstruction algorithm.

In some embodiments, the method 300 comprises reading out 330 a plurality of sets of image data at a detector, wherein each set of image data comprises a plurality of rows. The method may comprise reading out 330, by the detector, each row of each of the plurality of sets of image data successively. In particular, the detector may read out, successively, each row of a first set of the plurality of sets of image data. The detector may commence reading out, successively, each row of the second set of the plurality of sets of image data once all rows of the first set of the plurality of sets of image data have been read out. This may be repeated consecutively for all n sets of the plurality of sets of image data.

The method may comprise assigning 340 a rotation angle to each row of each of the plurality of sets of image data. Each row of each of the plurality of sets of image data and the respective assigned rotation angles may then be output 350 to an image reconstruction algorithm. The image reconstruction algorithm may reconstruct a 3D image of the subject or part of the subject. Additionally or alternatively, the image reconstruction algorithm may reconstruct one or more 2D images of the subject or region of interest or part of the subject. An image reconstruction system may be provided, configured to reconstruct 3D images based at least in part on each of the rows of image data and the respective assigned rotation angles.

Each of steps 320, 330 and/or 340 may be iterated as many times as necessary before moving on to another step. Each of steps 320, 330 and/or 340 may be carried out for a given set of image data at least once, and then repeated in any order for another set of image data as many times as is necessary.

Each row of image data may be delivered to the image reconstruction algorithm after any one of steps 320, 330 and/or 340, and/or as a batch of image data after completion of all iterations of steps 320, 330 and/or 340. The assigned rotation angles for each row of image data may be delivered to the image reconstruction algorithm together with, before or after the respective row of image data delivered in any manner described herein.

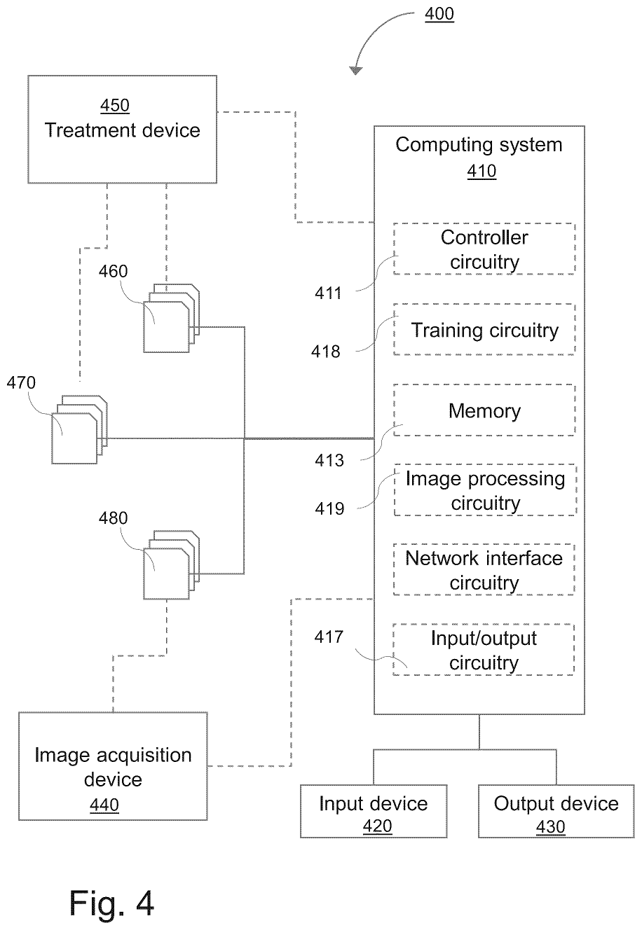

FIG. 4 illustrates a block diagram of one implementation of an imaging system 400. The imaging system 400 comprises a computing system 410 within which a set of instructions, for causing the computing system 410 to perform any one or more of the methods discussed herein, may be executed.

The computing system 410 shall be taken to include any number or collection of machines, e.g. computing device(s), that individually or jointly execute a set (or multiple sets) of instructions to perform any one or more of the methods discussed herein. That is, hardware and/or software may be provided in a single computing device, or distributed across a plurality of computing devices in the computing system. In some implementations, one or more elements of the computing system may be connected (e.g. networked) to other machines, for example in a Local Area Network (LAN), an intranet, an extranet, or the Internet. One or more elements of the computing system may operate in the capacity of a server or a client machine in a client-server network environment, or as a peer machine in a peer-to-peer (or distributed) network environment. One or more elements of the computing system may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine.

The computing system 410 includes controller circuitry 411 and a memory 413 (e.g., read-only memory (ROM), flash memory, dynamic random access memory (DRAM) such as synchronous DRAM (SDRAM) or Rambus DRAM (RDRAM), etc.). The memory 413 may comprise a static memory (e.g., flash memory, static random access memory (SRAM), etc.), and/or a secondary memory (e.g., a data storage device), which communicate with each other via a bus (not shown).

Controller circuitry 411 represents one or more general-purpose processors such as a microprocessor, central processing unit, accelerated processing units, or the like. More particularly, the controller circuitry 411 may comprise a complex instruction set computing (CISC) microprocessor, reduced instruction set computing (RISC) microprocessor, very long instruction word (VLIW) microprocessor, processor implementing other instruction sets, or processors implementing a combination of instruction sets. Controller circuitry 411 may also include one or more special-purpose processing devices such as an application specific integrated circuit (ASIC), a field programmable gate array (FPGA), a digital signal processor (DSP), a network processor, or the like. One or more processors of the controller circuitry may have a multicore design. Controller circuitry 411 is configured to execute the processing logic for performing the operations and steps discussed herein.

The computing system 410 may further include a network interface circuitry 418. The computing system 410 may be communicatively coupled to an input device 420 and/or an output device 430, via input/output circuitry 417. In some implementations, the input device 420 and/or the output device 430 may be elements of the computing system 410. The input device 420 may include an alphanumeric input device (e.g., a keyboard or touchscreen), a cursor control device (e.g., a mouse or touchscreen), an audio device such as a microphone, and/or a haptic input device. The output device 430 may include an audio device such as a speaker, a video display unit (e.g., a liquid crystal display (LCD) or a cathode ray tube (CRT)), and/or a haptic output device. In some implementations, the input device 420 and the output device 430 may be provided as a single device, or as separate devices.

In some implementations, the computing system 410 may comprise image processing circuitry 419. Image processing circuitry 419 may be configured to process image data such as image data obtained at the detector 200 upon reception of radiation, any of a plurality of sets of image data obtained at the detector 200, individual rows of image data and/or other image data obtained using a CBCT imaging system according to any of FIGS. 1-3. Image processing circuitry 419 may be configured to process, or pre-process, image data. For example, image processing circuitry 419 may convert received or obtained image data into a particular format, size, resolution or the like. In some implementations, image processing circuitry 419 may be combined with controller circuitry 411. Image data received by image processing circuitry 419 may comprise image data obtained by the detector 200 upon reception of radiation.

The imaging system 400 comprises an image acquisition device 440 such as CBCT imaging system 100. In exemplar embodiments wherein a radiotherapy device comprises an imaging system such as the CBCT imaging system 100, the imaging system 400 may further comprise a treatment device 450. The image acquisition device 440, such as CBCT imaging system 100, and the treatment device 450 may be provided as a single device.

The image acquisition device 440 may comprise the main image acquisition components of the imaging system, such as a source 110 and detector 200 and optionally any other components described with reference to CBCT imaging system 100 according to FIGS. 1A and 1B.

In some implementations, treatment device 450 is configured to perform imaging, for example in addition to providing treatment and/or during treatment. The treatment device 450 may comprise the main radiation delivery components of a radiotherapy system, such as the linac.

Image acquisition device 440 may be configured to perform computed tomography (CT), cone-beam computed tomography (CBCT), tomosynthesis or other imaging modalities.

Image acquisition device 440, for example the CBCT imaging system 100, may be configured to obtain, read out or output and/or deliver image data 480, which may be accessed by computing system 410. Treatment device 450 may be configured to output treatment data 460, which may be accessed by computing system 410.

Computing system may be configured to access, receive or obtain treatment data 460, planning data 470 and/or image data 480. Treatment data 460 may be obtained from an internal data source (e.g. from memory 413) or from an external data source, such as treatment device 450 or an external database. Planning data 460 may be obtained from memory 413 and/or from an external source, such as a planning database. Planning data 470 may comprise information obtained from one or more of the image acquisition device 440 and the treatment device 450. In some embodiments, the assigning a rotation angle of step 330 may depend on planning data 470 and/or treatment data 460. Additionally or alternatively, the obtaining image data of step 320 may depend on planning data and/or treatment data 460.

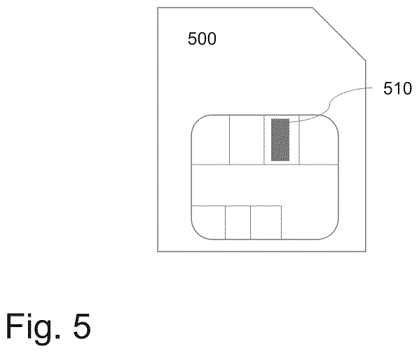

The various methods described above may be implemented by a computer program. The computer program may include computer code (e.g. instructions) 510 arranged to instruct a computer to perform the functions of one or more of the various methods described above. The steps of the methods described above may be performed in any suitable order. For example, step 330 of method 300 may be performed before, after, simultaneously or substantially simultaneously with step 340. Additionally or alternatively, step 340 of method 300 may be performed before, after, simultaneously or substantially simultaneously with step 320. The computer program and/or the code 510 for performing such methods may be provided to an apparatus, such as a computer, on one or more computer readable media or, more generally, a computer program product 500, depicted in FIG. 5. The computer readable media may be transitory or non-transitory. The one or more computer readable media 500 could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD. The instructions 510 may also reside, completely or at least partially, within the memory 413 and/or within the controller circuitry 411 during execution thereof by the computing system 410, the memory 413 and the controller circuitry 411 also constituting computer-readable storage media.

In an implementation, the modules, components and other features described herein can be implemented as discrete components or integrated in the functionality of hardware components such as ASICS, FPGAs, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may comprise a special-purpose processor, such as an FPGA or an ASIC. A hardware component may also include programmable logic or circuitry that is temporarily configured by software to perform certain operations.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “obtaining”, “assigning”, “reading out”, “delivering”, “receiving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

It is to be understood that the above description is intended to be illustrative, and not restrictive. Many other implementations will be apparent to those of skill in the art upon reading and understanding the above description. Although the present disclosure has been described with reference to specific example implementations, it will be recognised that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. An imaging system comprising:
a source of radiation rotatable around a subject and configured to deliver imaging radiation to the subject from a plurality of angles; and
a detector comprising a plurality of rows of detector elements, wherein the imaging system is configured to:
receive the radiation;
read out image data representing received radiation successively from each row of the detector;
assign a rotation angle to each row of the image data; and
output each row of the image data and the respective assigned rotation angle to an image reconstruction algorithm for reconstruction of a three-dimensional image of the subject.

2. The imaging system of claim 1, wherein the detector is rotatable in synchrony with the source.

3. The imaging system of claim 1, wherein source is configured to continuously deliver the radiation while rotating through the plurality of angles.

4. The imaging system of claim 1, wherein source is configured to continuously rotate through the plurality of angles.

5. The imaging system of claim 1, wherein the image data forms a first set of image data and, wherein once the first set of image data has been read out, the detector is configured to receive subsequently delivered radiation and read out image data representing the received subsequently delivered radiation successively from each row of the detector to form a second set of image data.

6. The imaging system of claim 5, wherein the detector is configured to read out a plurality of sets of image data, wherein each set of image data comprises a plurality of rows.

7. The imaging system of claim 1, wherein the source is configured to rotate though 360 degrees around the subject.

8. The imaging system of claim 1, wherein the radiation comprises a cone-shaped beam of radiation.

9. The imaging system of claim 1, wherein the radiation comprises x-ray radiation.

10. The imaging system of claim 1, wherein the rotation angle corresponds to a particular angular position, or angular range, of the source around the subject.

11. The imaging system of claim 1, wherein the detector is positioned diametrically opposite the source.

12. The imaging system of claim 1, wherein the source and detector are coupled to a rotatable gantry.

13. The imaging system of claim 1, further comprising:
an image reconstruction system configured to reconstruct the three-dimensional image based at least in part on each row of image data and the respective assigned rotation angles.

14. A detector comprising:
a plurality of rows of detector elements and configured to:
receive radiation from a source;
read out image data representing received radiation successively from each row of the detector;
assign a rotation angle to each row of the image data; and
output each row of the image data and the respective assigned rotation angle to an image reconstruction algorithm for reconstruction of a three-dimensional image of a subject.

15. A method for imaging a subject using an imaging system, the method comprising:
receiving, at a detector comprising a plurality of rows of detector elements, radiation delivered from a source to a subject;
reading out, from the detector, image data representing the received radiation successively from each of the plurality of rows;
assigning a rotation angle to each row of the image data; and
outputting each row of the image data and the respective assigned rotation angles to an image reconstruction algorithm for reconstruction of a three-dimensional image of the subject.

16. The method of claim 15, further comprising:
rotating the source around a subject and delivering imaging radiation to the subject from a plurality of angles.

17. The method of claim 15, further comprising:
rotating the detector, in synchrony with the source, around a subject.

18. The method of claim 15, wherein the source is configured to continuously deliver the radiation while rotating through a plurality of angles.

19. The method of claim 15, wherein the image data forms a first set of image data and, wherein once the first set of image data has been read out, the method further comprises:
receiving subsequently delivered radiation and reading out image data representing the received subsequently delivered radiation successively from each of the plurality of rows to form a second set of image data.

20. The method of claim 15, wherein obtaining image data comprises obtaining a plurality of sets of image data, wherein each set of image data comprises a plurality of rows.

21. The method of claim 15, wherein the radiation comprises a cone-shaped beam of radiation or wherein the radiation comprises x-ray radiation.

22. The method of claim 15, wherein the rotation angle corresponds to a particular angular position or an angular range of the source around the subject.

23. The method of claim 15, wherein the detector is positioned diametrically opposite the source.

24. The method of claim 15, wherein the source and detector are coupled to a rotatable gantry.

25. The method of claim 24, further comprising:
rotating the rotatable gantry about the subject.

26. The method of claim 15, wherein the reconstruction of the three-dimensional image is based at least in part on each of the plurality of rows of image data and the respective assigned rotation angles.
